(19) United States
(12) Patent Application Publication
(10) Pub. No.: US 2024/0226353 A1
(43) Pub. Date: Jul. 11, 2024

(54) Devices Configured to Provide Disinfection

(71) Applicant: Vyv, Inc., Latham, NY (US)

(72) Inventors: Cori Winslow, Rensselaer, NY (US); Jonas Ciemny, Colonie, NY (US); Edward Kiegle, Scotia, NY (US)

(21) Appl. No.: 18/404,419

(22) Filed: Jan. 4, 2024

Related U.S. Application Data
(60) Provisional application No. 63/478,591, filed on Jan. 5, 2023.

(51) Int. Cl. A61L 2/10; A61L 2/08; A61L 2/26; D06F 23/02; D06F 33/43; D06F 34/06; D06F 34/20; D06F 37/04; D06F 37/26; D06F 39/14; D06F 103/24; D06F 105/54; D06F 105/56

(52) U.S. Cl. CPC A61L 2/10; A61L 2/084; A61L 2/26; D06F 23/02; D06F 33/43; D06F 34/06; D06F 34/20; D06F 37/04; D06F 37/266; D06F 39/14; A61L 2202/11; A61L 2202/14; D06F 2103/24; D06F 2105/54; D06F 2105/56

(57) ABSTRACT

A system or device configured to disinfect an appliance may include at least one or more light emitters configured to emit disinfecting light. The system may be further configured to direct the disinfecting light to illuminate a surface of an appliance.

### CROSS REFERENCE TO RELATED APPLICATIONS

This application claims the benefit of U.S. Provisional Application No. 63/478,591 filed on Jan. 5, 2023. The above-referenced application is hereby incorporated by reference in its entirety.

### BACKGROUND

Many consumer appliances may be inhabited by harmful microorganisms such as bacteria, mold, fungi, etc. In some examples, microbial contamination may result from normal usage of an appliance. For example, an appliance that utilizes water may contain bacteria within it due to stagnant water. Many appliances, such as washing machines, are in contact with water that may create an environment for the growth of bacteria, mold, and other microorganisms. Many microorganisms may create unpleasant odors within consumer devices and appliances. As another example, user interaction with an appliance may result in microbial contamination (e.g., on an appliance handle, a door handle). Microorganisms may transfer to other users, through contact of the same appliances, and may result in illness. Harmful bacteria such as Escherichia coli (E. coli), Salmonella, Methicillin-resistant Staphylococcus aureus (MRSA), and Clostridium difficile may be found on many devices and appliances, and may result in a user illness or bacterial transmission. Various household appliances are susceptible to bacterial contamination. Appliances such as washing machines, for example, may contain a level of bacteria that is above a level that may be considered to be safe. In absence of regular cleaning, bacteria may establish and propagate within the washing machine. This may result in unsightly build-up, bad odors, illness, etc. A specific location within a front load washing machine that may comprise bacterial, and microorganism build up is the door gasket.

Disinfection (e.g., disinfection of surfaces in various environments) may be accomplished using different techniques. One technique may be manual cleaning with disinfecting chemical cleaners or soaps. Chemical cleaners may provide only intermittent disinfection, thereby allowing harmful microorganisms to build up between cleanings. Another technique may be ultraviolet (UV) light exposure. For example, some disinfecting systems may transmit UV light onto surfaces for disinfection. Exposure to UV light may be harmful for humans and animals, and appropriate steps may be needed to minimize such exposure. UV light may be turned off, for example, when exposure (e.g., to a user) is anticipated. UV light disinfection systems may involve complex controls to prevent direct exposure to humans to facilitate such safety mechanisms. Exposure to UV light may degrade plastics and rubbers (e.g., washing machine gaskets, etc.).

Larger spaces (e.g., entire rooms) may be disinfected as part of general illumination systems. A UV light, violet light, disinfecting light, or white light comprising a certain proportion of disinfecting light, for example, may be used. General overhead illumination may not be applicable for appliances because light may not necessarily be able to make sufficient contact with infected surfaces within an appliance (e.g., inside of a washing machine, and/or the like). Other challenges for providing disinfection to appliances may include designing a disinfection system for interior and/or exterior surfaces with irregular shapes, and/or comprising objects that are not originally intended to have such a disinfection system associated therewith.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and methods are also within the scope of the disclosure.

According to one aspect of the present disclosure, a device may be configured to provide disinfection of other objects and may include light emitters capable of emitting disinfecting light. In some examples, the disinfecting light may exit the device and may illuminate a surface of another object. In other examples, the object is an appliance. In still other examples, the device may be attached to the other object in a specific location to target specific surfaces on the object. In yet another example, the device may target a front load washing machine. In one example, the device may target a front load washing machine door gasket.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIGS. 1A-1B illustrate an example device configured to provide disinfection to other objects as disclosed herein;

FIG. 2 illustrates another example device configured to provide disinfection to other objects as disclosed herein;

FIGS. 3A-3B illustrate another example device configured to provide disinfection to other objects as disclosed herein;

FIG. 4 illustrates an example light emitter as disclosed herein;

FIG. 5 illustrates another example light emitter as disclosed herein;

FIGS. 6A-6B illustrate another example light emitter as disclosed herein;

FIG. 7 illustrates another example light emitter as disclosed herein;

FIGS. 8A-8B illustrate an example lens as disclosed herein;

FIG. 9 illustrates another example device configured to provide disinfection to other objects as disclosed herein;

FIG. 10A-10B illustrates another example device configured to provide disinfection to other objects as disclosed herein;

FIG. 11 illustrate another example device configured to provide disinfection to other objects as disclosed herein; and

FIG. 12 illustrates another example device configured to provide disinfection to other objects as disclosed herein.

FIG. 13A-13B illustrates another example device configured to provide disinfection to other objects as disclosed herein.

FIGS. 14A-14B illustrate another example device configured to provide disinfection to other objects as disclosed herein.

### DETAILED DESCRIPTION

Wavelengths of visible light in the violet range, 380-420 nanometers (nm) (e.g., 405 nm), may have a lethal effect on microorganisms. As used herein, the term “microorganisms” encompasses at least viruses (including enveloped and non-enveloped viruses), bacteria (including gram-positive and gram-negative bacteria), bacterial endospores, yeasts, molds, and filamentous fungi. For example, Escherichia coli (E. coli), Salmonella, Methicillin-resistant Staphylococcus aureus (MRSA), and Clostridium difficile may be susceptible to 380-420 nm light. Such wavelengths may initiate a photoreaction within non-iron porphyrin molecules found in some microorganisms. The non-iron porphyrin molecules may be photoactivated and may react with other cellular components to produce Reactive Oxygen Species (ROS). ROS may cause irreparable cell damage and eventually destroy, kill, or otherwise inactivate cells of some microorganisms. Non-iron porphyrins are specific to microorganisms only, therefore because humans, plants, and/or animals do not contain these same non-iron porphyrin molecules, this technique may be completely safe for human, plant, and animal exposure. Light in the 380-420 nm wavelength range may be effective against every type of bacteria, although it may take different amounts of time or dosages depending upon the species. 380-420 nm light (e.g., 405 nm), may be effective against all gram-negative and gram-positive bacteria to some extent over a period of time. It can also be effective against many varieties of fungi.

In some examples, visible light in the violet range, 380-420 nanometers (nm) (e.g., 405 nm), may decrease viral load on a surface. Viruses may rely on surface bacteria, yeast, mold, or fungi as hosts. By decreasing surface bacteria, yeast, mold, or fungi count, for example, by using 380-420 nm light, the viral load may also be decreased. In some examples, viruses may be susceptible to reactive oxygen species. Viral load may decrease when the viruses are surrounded by a medium that can produce reactive oxygen species to inactivate viruses. In some examples, the medium may comprise fluids or droplets that comprise bacteria or other particles that produce oxygen reactive species. In some examples, the medium may comprise respiratory droplets, saliva, feces, organic rich media, and/or blood plasma.

Example methods, devices, and systems described herein may use visible light (e.g., 380 nm-420 nm wavelength light, and/or a specific wavelength in the wavelength range) for disinfection. Visible light disinfection may be used for continuous, efficient, and effective decontamination of various surfaces, devices, and/or appliances. Visible light disinfection may be simultaneous with normal operation and without interruption of other functions of the devices and/or appliances. Daily and/or terminal cleaning procedures may be supplemented with visible light disinfection to maintain cleanliness between such cleaning procedures. Visible light disinfection may be used, for example, to combat any new sources of contamination and/or to reduce growth rates of microorganisms that may be left behind after typical cleaning procedures.

In some examples, inactivation, in relation to microorganism death, may include control and/or reduction in microorganism colonies or individual cells when exposed to disinfecting light for a certain duration. Light may be utilized for inactivation using a peak wavelength of light, or in some examples, multiple peak wavelengths, in a range of approximately 380 nm to 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. According to one example, peak wavelength may include, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

Such light may inactivate viruses. Such light may damage viral capsids, surface proteins, nucleic acids, and also lead to the degradation of the nucleic acids. Destruction of nucleic acids and genomes may prevent replication function in host cells leading to loss of infectivity. Unsaturated lipids and alterations of envelope proteins may cause conformational changes in the viral structure that alters viral interactions with host cell receptors. Protein mediated binding, injection or replication functions may be impaired. Significant changes in molecular mass and charge of proteins may occur, which may hinder viral entry and cytopathic effects.

The electromagnetic spectrum may be harnessed within devices, systems, and apparatuses to utilize its functions for the benefit of humans/animals. Most portions of the electromagnetic spectrum are not visible to the human eye with the exception of the visible light spectrum within the range of approximately 380 nm to 750 nm. The ultraviolet spectrum comprises the energy within the range of approximately 100 nm to 400 nm and is generally not visible to humans. Light comprising wavelengths that cause microbial inactivation or disinfection may be referred to as “disinfecting light.” Disinfecting light may be emitted by one or more light emitters. There may be a minimum irradiance required to hit the surface to cause microbial inactivation. A target irradiance may be required on at least a portion of the surface. A minimum irradiance of disinfecting light (e.g., in the 380-420 nm wavelength) on a surface may cause microbial inactivation. For example, a minimum irradiance of 0.02 milliwatts per square centimeter (mW/cm²) may cause microbial inactivation on a surface over time. In some examples, an irradiance of 0.05 mW/cm² may inactivate microorganisms on a surface, but higher values such as 0.1 mW/cm², 0.5 mW/cm², 1 mW/cm², or 2 mW/cm² may be used for quicker microorganism inactivation. In some examples, even higher irradiances may be used over shorter periods of time, e.g., 3 to 10 mW/cm². In other examples, a target irradiance may be, for example, at least, greater than, less than, equal to, or any number in between about 0.01 mW/cm², 0.02 mW/cm², 0.03 mW/cm², 0.04 mW/cm², 0.05 mW/cm², 0.06 mW/cm², 0.07 mW/cm², 0.08 mW/cm², 0.09 mW/cm², 0.1 mW/cm², 0.1 mW/cm², 0.2 mW/cm², 0.3 mW/cm², 0.4 mW/cm², 0.5 mW/cm², 0.6 mW/cm², 0.7 mW/cm², 0.8 mW/cm², 0.9 mW/cm², 1.0 mW/cm², 1.1 mW/cm², 1.2 mW/cm², 1.3 mW/cm², 1.4 mW/cm², 1.5 mW/cm², 1.6 mW/cm², 1.7 mW/cm², 1.8 mW/cm², 1.9 mW/cm², 2.0 mW/cm², 2.1 mW/cm², 2.2 mW/cm², 2.3 mW/cm², 2.4 mW/cm², 2.5 mW/cm², 2.6 mW/cm², 2.7 mW/cm², 2.8 mW/cm², 2.9 mW/cm², 3.0 mW/cm², 3.1 mW/cm², 3.2 mW/cm², 3.3 mW/cm², 3.4 mW/cm², 3.5 mW/cm², 3.6 mW/cm², 3.7 mW/cm², 3.8 mW/cm², 3.9 mW/cm², 4.0 mW/cm², 4.1 mW/cm², 4.2 mW/cm², 4.3 mW/cm², 4.4 mW/cm², 4.5 mW/cm², 4.6 mW/cm², 4.7 mW/cm², 4.8 mW/cm², 4.9 mW/cm², 5.0 mW/cm², 5.1 mW/cm², 5.2 mW/cm², 5.3 mW/cm², 5.4 mW/cm², 5.5 mW/cm², 5.6 mW/cm², 5.7 mW/cm², 5.8 mW/cm², 5.9 mW/cm², 6.0 mW/cm², 6.1 mW/cm², 6.2 mW/cm², 6.3 mW/cm², 6.4 mW/cm², 6.5 mW/cm², 6.6 mW/cm², 6.7 mW/cm², 6.8 mW/cm², 6.9 mW/cm², 7.0 mW/cm², 7.1 mW/cm², 7.2 mW/cm², 7.3 mW/cm², 7.4 mW/cm², 7.5 mW/cm², 7.6 mW/cm², 7.7 mW/cm², 7.8 mW/cm², 7.9 mW/cm², 8.0 mW/cm², 8.1 mW/cm², 8.2 mW/cm², 8.3 mW/cm², 8.4 mW/cm², 8.5 mW/cm², 8.6 mW/cm², 8.7 mW/cm², 8.8 mW/cm², 8.9 mW/cm², 9.0 mW/cm², 9.1 mW/cm², 9.2 mW/cm², 9.3 mW/cm², 9.4 mW/cm², 9.5 mW/cm², 9.6 mW/cm², 9.7 mW/cm², 9.8 mW/cm², 9.9 mW/cm², and 10.0 mW/cm². Example light emitters disclosed herein may be configured to produce light with such irradiances at any given surface.

In some examples, an average irradiance is targeted across a surface or at least a portion of a surface. The average may comprise an average of multiple measurement points taken across at least a portion of the surface. Irradiance measurements may range from 0 mW/cm² to 100 mW/cm² in some examples. In some examples, the target average irradiance may be 0.05 mW/cm². In some examples, the target average irradiance may be 1 mW/cm². In some examples, the target average irradiance may be any value within the range of 0.02 to 2 mW/cm². In some examples, the target average irradiance may be any value within the range of 0.02 to 5 mW/cm². In still another example, the average irradiance may be, for example, at least, greater than, less than, equal to, or any number in between about 0.01 mW/cm², 0.02 mW/cm², 0.03 mW/cm², 0.04 mW/cm², 0.05 mW/cm², 0.06 mW/cm², 0.07 mW/cm², 0.08 mW/cm², 0.09 mW/cm², 0.1 mW/cm², 0.1 mW/cm², 0.2 mW/cm², 0.3 mW/cm², 0.4 mW/cm², 0.5 mW/cm², 0.6 mW/cm², 0.7 mW/cm², 0.8 mW/cm², 0.9 mW/cm², 1.0 mW/cm², 1.1 mW/cm², 1.2 mW/cm², 1.3 mW/cm², 1.4 mW/cm², 1.5 mW/cm², 1.6 mW/cm², 1.7 mW/cm², 1.8 mW/cm², 1.9 mW/cm², 2.0 mW/cm², 2.1 mW/cm², 2.2 mW/cm², 2.3 mW/cm², 2.4 mW/cm², 2.5 mW/cm², 2.6 mW/cm², 2.7 mW/cm², 2.8 mW/cm², 2.9 mW/cm², 3.0 mW/cm², 3.1 mW/cm², 3.2 mW/cm², 3.3 mW/cm², 3.4 mW/cm², 3.5 mW/cm², 3.6 mW/cm², 3.7 mW/cm², 3.8 mW/cm², 3.9 mW/cm², 4.0 mW/cm², 4.1 mW/cm², 4.2 mW/cm², 4.3 mW/cm², 4.4 mW/cm², 4.5 mW/cm², 4.6 mW/cm², 4.7 mW/cm², 4.8 mW/cm², 4.9 mW/cm², 5.0 mW/cm², 5.1 mW/cm², 5.2 mW/cm², 5.3 mW/cm², 5.4 mW/cm², 5.5 mW/cm², 5.6 mW/cm², 5.7 mW/cm², 5.8 mW/cm², 5.9 mW/cm², 6.0 mW/cm², 6.1 mW/cm², 6.2 mW/cm², 6.3 mW/cm², 6.4 mW/cm², 6.5 mW/cm², 6.6 mW/cm², 6.7 mW/cm², 6.8 mW/cm², 6.9 mW/cm², 7.0 mW/cm², 7.1 mW/cm², 7.2 mW/cm², 7.3 mW/cm², 7.4 mW/cm², 7.5 mW/cm², 7.6 mW/cm², 7.7 mW/cm², 7.8 mW/cm², 7.9 mW/cm², 8.0 mW/cm², 8.1 mW/cm², 8.2 mW/cm², 8.3 mW/cm², 8.4 mW/cm², 8.5 mW/cm², 8.6 mW/cm², 8.7 mW/cm², 8.8 mW/cm², 8.9 mW/cm², 9.0 mW/cm², 9.1 mW/cm², 9.2 mW/cm², 9.3 mW/cm², 9.4 mW/cm², 9.5 mW/cm², 9.6 mW/cm², 9.7 mW/cm², 9.8 mW/cm², 9.9 mW/cm², and 10.0 mW/cm².

In some examples, light for microbial inactivation or disinfecting light may include radiometric energy sufficient to inactive at least one microorganism population, or in some examples, a plurality of microorganism populations. One or more light emitters(s) may emit some minimum amount of radiometric energy (e.g., 20 mW) measured from 380-420 nm light. In one example, one or more light emitter(s) may emit some minimum amount of radiometric energy measured from, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In another example, one or more light emitter(s) may emit some minimum amount of radiometric energy measured from, for example, at least, greater than, less than, equal to, or any number in between about 10 mW, 15 mW, 20 mW, 25 mW, 30 mW, 35 mW, 40 mW, 45 mW, 50 mW, 55 mW, 60 mW, 65 mW, 70 mW, 75 mW, 80 mW, 85 mW, 90 mW, 95 mW, 100 mW, 105 mW, 110 mW, 115 mW, 120 mW, 125 mW, 130 mW, 135 mW, 140 mW, 145 mW, 150 mW, 155 mW, 160 mW, 165 mW, 170 mW, 175 mW, 180 mW, 185 mW, 190 mW, 195 mW, 200 mW, 205 mW, 210 mW, 215 mW, 220 mW, 225 mW, 230 mW, 235 mW, 240 mW, 245 mW, 250 mW, 255 mW, 260 mW, 265 mW, 270 mW, 275 mW, 280 mW, 285 mW, 290 mW, 295 mW, 300 mW, 305 mW, 310 mW, 315 mW, 320 mW, 325 mW, 330 mW, 335 mW, 340 mW, 345 mW, 350 mW, 355 mW, 360 mW, 365 mW, 370 mW, 375 mW, 380 mW, 385 mW, 390 mW, 395 mW, 400 mW, 405 mW, 410 mW, 415 mW, 420 mW, 425 mW, 430 mW, 435 mW, 440 mW, 445 mW, 450 mW, 455 mW, 460 mW, 465 mW, 470 mW, 475 mW, 480 mW, 485 mW, 490 mW, 495 mW, 500 mW, 505 mW, 510 mW, 515 mW, 520 mW, 525 mW, 530 mW, 535 mW, 540 mW, 545 mW, 550 mW, 555 mW, 560 mW, 565 mW, 570 mW, 575 mW, 580 mW, 585 mW, 590 mW, 595 mW, 600 mW, 605 mW, 610 mW, 615 mW, 620 mW, 625 mW, 630 mW, 635 mW, 640 mW, 645 mW, 650 mW, 655 mW, 660 mW, 665 mW, 670 mW, 675 mW, 680 mW, 685 mW, 690 mW, 695 mW, 700 mW, 705 mW, 710 mW, 715 mW, 720 mW, 725 mW, 730 mW, 735 mW, 740 mW, 745 mW, 750 mW, 755 mW, 760 mW, 765 mW, 770 mW, 775 mW, 780 mW, 785 mW, 790 mW, 795 mW, 800 mW, 805 mW, 810 mW, 815 mW, 820 mW, 825 mW, 830 mW, 835 mW, 840 mW, 845 mW, 850 mW, 855 mW, 860 mW, 865 mW, 870 mW, 875 mW, 880 mW, 885 mW, 890 mW, 895 mW, 900 mW, 905 mW, 910 mW, 915 mW, 920 mW, 925 mW, 930 mW, 935 mW, 940 mW, 945 mW, 950 mW, 955 mW, 960 mW, 965 mW, 970 mW, 975 mW, 980 mW, 985 mW, 990 mW, 995 mW, 1000 mW, 1005 mW, 1010 mW, 1015 mW, 1020 mW, 1025 mW, 1030 mW, 1035 mW, 1040 mW, 1045 mW, 1050 mW, 1055 mW, 1060 mW, 1065 mW, 1070 mW, 1075 mW, 1080 mW, 1085 mW, 1090 mW, 1095 mW, 1100 mW, 1105 mW, 1110 mW, 1115 mW, 1120 mW, 1125 mW, 1130 mW, 1135 mW, 1140 mW, 1145 mW, 1150 mW, 1155 mW, 1160 mW, 1165 mW, 1170 mW, 1175 mW, 1180 mW, 1185 mW, 1190 mW, 1195 mW, 1200 mW, 1205 mW, 1210 mW, 1215 mW, 1220 mW, 1225 mW, 1230 mW, 1235 mW, 1240 mW, 1245 mW, 1250 mW, 1255 mW, 1260 mW, 1265 mW, 1270 mW, 1275 mW, 1280 mW, 1285 mW, 1290 mW, 1295 mW, 1300 mW, 1305 mW, 1310 mW, 1315 mW, 1320 mW, 1325 mW, 1330 mW, 1335 mW, 1340 mW, 1345 mW, 1350 mW, 1355 mW, 1360 mW, 1365 mW, 1370 mW, 1375 mW, 1380 mW, 1385 mW, 1390 mW, 1395 mW, 1400 mW, 1405 mW, 1410 mW, 1415 mW, 1420 mW, 1425 mW, 1430 mW, 1435 mW, 1440 mW, 1445 mW, 1450 mW, 1455 mW, 1460 mW, 1465 mW, 1470 mW, 1475 mW, 1480 mW, 1485 mW, 1490 mW, 1495 mW, 1500 mW, 1505 mW, 1510 mW, 1515 mW, 1520 mW, 1525 mW, 1530 mW, 1535 mW, 1540 mW, 1545 mW, 1550 mW, 1555 mW, 1560 mW, 1565 mW, 1570 mW, 1575 mW, 1580 mW, 1585 mW, 1590 mW, 1595 mW, 1600 mW, 1605 mW, 1610 mW, 1615 mW, 1620 mW, 1625 mW, 1630 mW, 1635 mW, 1640 mW, 1645 mW, 1650 mW, 1655 mW, 1660 mW, 1665 mW, 1670 mW, 1675 mW, 1680 mW, 1685 mW, 1690 mW, 1695 mW, 1700 mW, 1705 mW, 1710 mW, 1715 mW, 1720 mW, 1725 mW, 1730 mW, 1735 mW, 1740 mW, 1745 mW, 1750 mW, 1755 mW, 1760 mW, 1765 mW, 1770 mW, 1775 mW, 1780 mW, 1785 mW, 1790 mW, 1795 mW, 1800 mW, 1805 mW, 1810 mW, 1815 mW, 1820 mW, 1825 mW, 1830 mW, 1835 mW, 1840 mW, 1845 mW, 1850 mW, 1855 mW, 1860 mW, 1865 mW, 1870 mW, 1875 mW, 1880 mW, 1885 mW, 1890 mW, 1895 mW, 1900 mW, 1905 mW, 1910 mW, 1915 mW, 1920 mW, 1925 mW, 1930 mW, 1935 mW, 1940 mW, 1945 mW, 1950 mW, 1955 mW, 1960 mW, 1965 mW, 1970 mW, 1975 mW, 1980 mW, 1985 mW, 1990 mW, 1995 mW, and 2000 mW.

Dosage (measured in Joules/cm²) may be another metric for determining an appropriate irradiance for microbial inactivation over a period of time. Table 1 below shows example correlations between irradiance in mW/cm² and Joules/cm² based on different exposure times. These values are examples, and many others may be possible.

TABLE 1

| Irradiance (mW/cm²) | Exposure Time (hours) | Dosage (Joules/cm²) |
|---|---|---|
| 0.02 | 1 | 0.072 |
| 0.02 | 24 | 1.728 |
| 0.02 | 250 | 18 |
| 0.02 | 500 | 36 |
| 0.02 | 1000 | 72 |
| 0.05 | 1 | 0.18 |
| 0.05 | 24 | 4.32 |
| 0.05 | 250 | 45 |
| 0.05 | 500 | 90 |
| 0.05 | 1000 | 180 |
| 0.1 | 1 | 0.36 |
| 0.1 | 24 | 8.64 |
| 0.1 | 250 | 90 |
| 0.1 | 500 | 180 |
| 0.1 | 1000 | 360 |
| 0.5 | 1 | 1.8 |
| 0.5 | 24 | 43.2 |
| 0.5 | 250 | 450 |
| 0.5 | 500 | 900 |
| 0.5 | 1000 | 1800 |
| 1 | 1 | 3.6 |
| 1 | 24 | 86.4 |
| 1 | 250 | 900 |
| 1 | 500 | 1800 |
| 1 | 1000 | 3600 |
| 2 | 1 | 7.2 |
| 2 | 24 | 172.8 |
| 2 | 250 | 1800 |
| 2 | 500 | 3600 |
| 2 | 1000 | 7200 |
| 5 | 1 | 18 |
| 5 | 24 | 432 |
| 5 | 250 | 4500 |
| 5 | 500 | 9000 |
| 5 | 1000 | 18000 |

Microbial inactivation may comprise a target reduction in microorganism population(s) (e.g., 1- Log₁₀ reduction, 2- Log₁₀ reduction, 99% reduction, or the like). Table 2 shows example dosages recommended for the inactivation (measured as 1- Log₁₀ reduction in population) of different microorganism species using narrow spectrum 405 nm light.

Example dosages and other calculations shown herein may be determined based on laboratory settings. Real world applications may require dosages that may differ from example laboratory data. Other dosages of 380-420 nm (e.g., 405 nm) light may be used with other bacteria not listed below.

TABLE 2

| | Recommended Dose (J/cm²) for |
|---|---|
| Microorganism | 1-Log Reduction in Microorganism |
| Staphylococcus aureus | 20 |
| MRSA | 20 |
| Pseudomonas aeruginosa | 45 |
| Escherichia coli | 80 |
| Enterococcus faecalis | 90 |

Equation 1 may be used in order to determine irradiance, dosage, or time using one or more data points from Table 1 and Table 2:

$$\frac{Irradiance ( \frac{mW}{{cm}^{2}} )}{1000} * Time ( s ) = Dosage ( \frac{J}{{cm}^{2}} ) \qquad Equation 1$$

Irradiance may be determined based on dosage and time. For example, if a dosage of 30 Joules/cm² is recommended and the object desired to be disinfected is exposed to light overnight for 8 hours, the irradiance may be approximately 1 mW/cm². If a dosage of 50 Joules/cm² is recommended and the object desired to be disinfected is exposed to light for 48 hours, a smaller irradiance of only approximately 0.3 mW/cm² may be sufficient.

Time may be determined based on irradiance and dosage. For example, light emitter(s) may be configured to provide an irradiance of disinfecting energy (e.g., 0.05 mW/cm²) and a target bacteria may require a dosage of 20 Joules/cm² to kill the target bacteria. Disinfecting light at 0.05 mW/cm² may have a minimum exposure time of approximately 4.6 days to achieve the dosage of 20 Joules/cm². Dosage values may be determined by a target reduction in microorganisms. Once the microorganism count is reduced to a desired amount, disinfecting light may be continuously applied to keep the microorganism counts down.

Radiant power (e.g., radiometric power, optical output power, spectral power etc.), measured in Watts, is the total power emitted from a light source. Irradiance is the power per unit area on a surface at a distance away from the light source. In some examples, the target irradiance on a target surface from the light source may be 10 mW/cm². A 10 mW/cm² target irradiance may be provided, for example, by light emitter(s) with a total radiant power of 10 mW located 1 cm from the target surface. In another example, light emitter(s) may be located 5 cm from the target surface. With a target irradiance of 10 mW/cm², the light source may be configured to produce a radiant power approximately 250 mW. These calculations may be approximately based on the inverse square law, as shown in Equation 1, where the excitation light source may be assumed to be a point source, E is the irradiance, I is the radiant power, and r is the distance from the excitation light source to a target surface.

$$E ≅ \frac{I}{{r}^{2}} \qquad Equation 2$$

In some examples, different wavelengths of light may have different effects on different microorganisms. The tables below illustrate example data related to application of various wavelengths of light on various microorganisms. For example, tables 3-7 summarize the recommended dose response for the inactivation of microorganisms at different log levels when exposed to wavelengths of 405 nm, 222 nm and 254 nm light. Inactivation may comprise a target reduction in microorganism population(s) (e.g., 1- Log₁₀ reduction, 2- Log₁₀ reduction, 99% reduction, or the like).

Table 3 shows example dosages measured in J/cm² which may be used for the inactivation (at different log levels) of different microorganisms using 222 nm light.

TABLE 3

| | Recommended Dose (J/cm²) for | | | |
|---|---|---|---|---|
| | Reduction in Microorganisms at 222 nm | | | |
| Microorganism | 1-log | 2-log | 3-log | 4-1og |
| Staphylococcus | 9.3 × 10⁻³ | 1.15 × 10⁻² | 1.38 × 10⁻² | 1.78 × 10⁻² |
| aureus | | | | |
| Pseudomonas | 3.1 × 10⁻³ | 4.8 × 10⁻³ | 5.9 × 10⁻³ | 7.5 × 10⁻³ |
| aeruginosa | | | | |
| Aspergillus | 9 × 10⁻² | 0.220 | 0.325 | 0.430 |
| niger | | | | |

Table 4 shows example dosages measured in J/cm² which may be used for the inactivation (at different log levels) of different microorganisms using 254 nm light.

TABLE 4

| | Recommended Dose (J/cm²) for | | | |
|---|---|---|---|---|
| | Reduction in Microorganisms at 254 nm | | | |
| Microorganism | 1-log | 2-log | 3-log | 4-log |
| Staphylococcus | 4.4 × 10⁻³ | 6.0 × 10⁻³ | 7.3 × 10⁻³ | 9.5 × 10⁻³ |
| aureus | | | | |
| Streptococcus | 6.6 × 10⁻³ | 8.8 × 10⁻³ | 9.9 × 10⁻³ | 1.12 × 10⁻² |
| faecalis | | | | |
| Pseudomonas | 8 × 10⁻⁴ | 1.6 × 10⁻³ | 2.3 × 10⁻³ | 3.1 × 10⁻³ |
| aeruginosa | | | | |
| Escherichia coli | 3 × 10⁻³ | 4.8 × 10⁻³ | 6.7 × 10⁻³ | 8.4 × 10⁻³ |
| Aspergillus | 0.115 | 0.245 | 0.370 | 0.560 |
| niger | | | | |

Table 5 shows example dosages measured in J/cm² which may be used for the inactivation (at different log levels) of different microorganisms using 222 nm light.

TABLE 5

| | Recommended Dose (J/cm²) for | | | |
|---|---|---|---|---|
| | Reduction in Microorganisms at 222 nm | | | |
| Microorganism | Type | Reduction | Light dosage | Medium |
| Influenza A | Enveloped | 1 log | 1.3 × 10⁻³ | Airborne |
| | | 2 log | 2.6 × 10⁻³ | |
| | | 3 log | 3.8 × 10⁻³ | |
| HcoV 229-E | Enveloped | 1 log | 5.6 × 10⁻⁴ | Airborne |
| | | 2 log | 1.1 × 10⁻³ | |
| | | 3 log | 1.7 × 10⁻³ | |
| HcoV OCV3 | Enveloped | 1 log | 3.9 × 10⁻⁴ | Airborne |
| | | 2 log | 7.8 × 10⁻⁴ | |
| | | 3 log | 1.2 × 10⁻³ | |

Table 6 shows example dosages measured in J/cm² which may be used for the inactivation (at different log levels) of different microorganisms using 254 nm light.

TABLE 6

| | Recommended Dose (J/cm²) for | | | | |
|---|---|---|---|---|---|
| | Reduction in Microorganisms at 254 nm | | | | |
| Microorganism | Type | Reduction | | Light dosage | Medium |
| Influenza A | Enveloped | 1 | log | 1.04 × 10⁻³ | Airborne |
| | | 1.4 | log | 1.48 × 10⁻³ | |
| Influenza A | Enveloped | 4.08 log to 5.75 log | | 1.8 | Solid |
| SARS CoV | Enveloped | 3.4 log to 3.6 log | | 0.15 | Liquid |
| | | 4 | log | 1.4 | |
| SARS CoV | Enveloped | 4 | log | 0.12 | Solid |
| SARS CoV2 | Enveloped | 5.7 | log | 1.6 × 10⁻² | Liquid |
| MS₂ | Non-enveloped | 1 | log | 3.4-4.2 × 10⁻⁴ | Airborne |
| bacteriophage | | 2 | log | 8-9.1 × 10⁻⁴ | |
| MS₂ | Non-enveloped | 1 | log | 1.86-2.57 × 10⁻² | Liquid |
| bacteriophage | | 4 | log | 0.12 | |
| MS₂ | Non-enveloped | 1 | log | 3.2 × 10⁻³ | Solid |
| bacteriophage | | 3 log to 4 log | | 4.32-7.2 | |
| FCV | Non-enveloped | 1 | log | 5-6 × 10⁻³ | Liquid |
| | | 4 | log | 0.04 | |
| FCV | Non-Enveloped | 2.12 log-4.46 log | | 0.2 | Solid |
| Adenovirus type | Non-enveloped | 1 | log | 5.5 × 10⁻² | Liquid |
| 40 | | 2 | log | 0.105 | |
| | | 3 | log | 0.155 | |
| Rotavirus | Non-enveloped | 1 | log | 2.0 × 10⁻² | Liquid |
| | | 2 | log | 8.0 × 10⁻² | |
| | | 3 | log | 0.140 | |
| | | 4 | log | 0.2 | |
| Polio virus 1 | Non-enveloped | 1 | log | 7 × 10⁻³ | Liquid |
| | | 2 | log | 1.7 × 10⁻² | |
| | | 3 | log | 2.8 × 10⁻² | |
| | | 4 | log | 3.7 × 10⁻² | |
| Hepatitis A | Non-enveloped | 1 | log | 5.5 × 10⁻³ | Liquid |
| | | 2 | log | 9.8 × 10⁻³ | |
| | | 3 | log | 1.5 × 10⁻² | |
| | | 4 | log | 2.1 × 10⁻² | |
| Murine | Non-enveloped | 1 | log | 1 × 10⁻² | Liquid |
| norovirus | | | | | |

Table 7 shows example dosages measured in J/cm² which may be used for the inactivation (at different log levels) of different microorganisms using 405 nm light.

TABLE 7

| | Recommended Dose (J/cm²) for | | | |
|---|---|---|---|---|
| | Reduction in Microorganisms at 405 nm | | | |
| Microorganism | Type | Reduction | Light dosage | Medium |
| SARS CoV2 | Enveloped | 1 log | 3.9 × 10⁻⁴ | Airborne |
| phi6 | Enveloped | 1 log | 430 | Liquid |
| | | 3 log | 1300 | |
| Bacteriophage | Non-Enveloped | 3 log | 300 | Liquid |
| sigma C31 | | 5 log | 500 | |
| | | 7 log | 1400 | |
| FCV | Non-enveloped | 3.9 log | 2800 | Liquid |

In some examples, one or more of the light emitter(s) disclosed herein may inactivate microorganisms/pathogens with light having a peak wavelength of light, or in some examples, multiple peak wavelengths, in a range of approximately 380 nm to approximately 420 nm. For example, approximately 405 nm light may be used as the peak wavelength. It should be understood that any wavelength within 380 nm to 420 nm may be utilized, and that the peak wavelength may include a specific wavelength plus or minus approximately 5 nm. In some examples, one or more light emitter(s) may emit some minimum amount of radiometric energy measured from, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, one or more of the light emitter(s) disclosed herein may inactivate microorganisms/pathogens with light having a peak wavelength of light, or in some examples, multiple peak wavelengths, in a range of approximately 200 nm to approximately 380 nm, for example, approximately 254 nm light may be used as the peak wavelength. It should be understood that any wavelength within 200 nm to 380 nm may be utilized, and that the peak wavelength may include a specific wavelength plus or minus approximately 5 nm. Light sources may additionally be within the following ranges: 100-280 nm, 200-230 nm, and/or 380-420 nm including, for example, UVA, UVC, visible, 222 nm, 254 nm, 260-270 nm, 280 nm, and/or 405 nm peak wavelength. In another example, one or more of the light emitter(s) disclosed herein may inactivate microorganisms/pathogens with light having a peak wavelength of light, or in some examples, multiple peak wavelengths, in a range of, at least, greater than, less than, equal to, or any number in between about 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, the device disclosed herein is a device configured to provide disinfection to other objects. The device may comprise light emitters capable of emitting a disinfecting light, wherein the disinfecting light may have wavelengths within the range of 380 to 420 nanometers (nm). The disinfecting light may exit the device and illuminate a surface of another object, such as an appliance. The device may be attached to the other object in a specific location such that the light emitted from the device may target specific surfaces on the object. In some examples, the object the device is disinfecting may be a front load washing machine. In some examples, the door gasket of the front load washing machine may be the surface of the object to be disinfected by the device configured to provide disinfection.

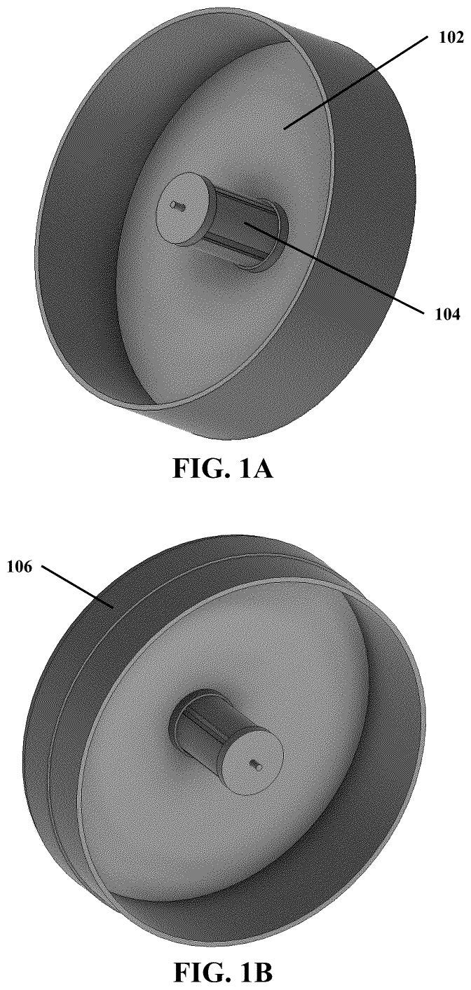

FIG. 1A demonstrates an example component 102 on which device 104 is located. Device 104 may be configured to provide disinfection to other objects. Component 106 of FIG. 1B may be a component disposed on or attached to component 102. Component 102 may comprise of multiple assembled components. Component 106 may comprise a surface. In some examples, component 106 is a gasket. Component 106 may be a door gasket. In some examples, component 102 is the door assembly of a washing machine. In some examples, device 104 is located on, installed on, attached to, mounted to, or otherwise coupled to component 102. In some examples, device 104 is located on, installed on, attached to, mounted to, or otherwise coupled to a different component than component 102.

In some examples, component 102 is a circular shape as shown in FIG. 1A and FIG. 1B. Component 102 may have other components attached to it to create a full assembly. In some examples, component 102 may be a shape other than a circle, such as a rectangle or irregular shape. Component 102 may have a circular profile. In some examples, component 102 may have a thickness within the range of 0.08 in and 6 in.

In some examples, component 102 may be transparent or translucent such that it allows light and/or disinfecting light to transmit through it. In some examples, component 102 may be a portion of a door. In some examples, component 102 may be comprised of glass. Component 102 may allow light to pass through it. In some examples, component 102 may allow for disinfecting light within the wavelengths of 380 to 420 nm to pass through it. In some examples, component 102 may be comprised of a plastic or polymer material. In some examples, component 102 may be opaque. Component 102 may have transparent or translucent lenses or windows embedded within it.

Component 102 may be domed in shape and/or may be concaved. In some examples, component 102 may be a component of a larger product and/or assembly such as an appliance. In some examples, component 102 may be part of a door of an appliance such as a washing machine.

In some examples, component 102 may have magnetic characteristics. In some examples, component 102 may be compatible with suction cups. In some examples, device 104 may be removably attached to component 102 via suction cups, magnets, snapping mechanisms, spring clip mechanisms, mating mounting bracket components, removable hardware, adhesives, etc. Component 102 may have integrated features which allow for mounting device 104 to it.

In some examples, component 106 of FIG. 1B may interface with component 102. Component 106 may be located on, installed on, attached to, mounted to, or otherwise coupled to component 102. In some examples, component 106 may be comprised of a flexible material. Component 106 may be comprised of rubber. In some examples, component 106 may be a gasket or a portion of a gasket. Component 106 may be comprised of the material EDPM. In some examples, component 106 may be a different shape or form than shown in FIG. 1B. In some examples, component 106 may have a different profile than shown in FIG. 1B. In some examples, component 106 may be a revolved ring shape with a profile shape. In some examples, the profile shape of component 106 may vary depending on the application. In some examples, component 106 may be rigidly fixed or flexibly fixed in place within a larger assembly.

In some examples, component 106 may be exposed to water. In some examples, component 106 may comprise undesirable microbial build-up. In some examples, component 106 may water seal component 102 against another component, assembly, or appliance. In some examples, component 106 may be opaque. In some examples, component 106 may be transparent or translucent. In some examples, component 106 may allow for light to pass through it. In some examples, component 106 may allow for disinfecting light within the wavelengths of 380 to 420 nm to pass through it.

In some examples, component 102 may be a part of an appliance such as a washing machine. Component 102 may be a portion of a door on a front load washing machine. In some examples, component 106 may be a door gasket on a front load washing machine. In some examples, component 102 may interface with component 106 but may not be attached. In some examples, component 106 may interface with other components or objects not shown in FIG. 1B.

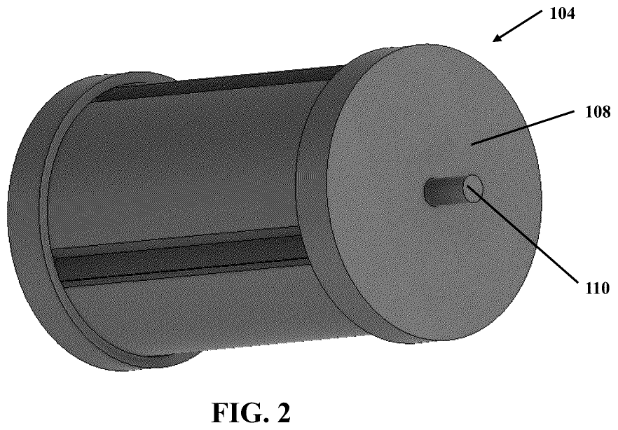

FIG. 2 demonstrates device 104 uninstalled. In some examples, device 104 may be cylindrical, triangular, or rectangular in shape. In some examples, device 104 may have different profile geometry than shown in FIG. 2. In some examples, device 104 may be a light fixture. In some examples, device 104 may comprise end cap 108 on one end, both ends, or neither end. Device 104 may have a hole in at least one end cap 108 for wire 110 to exit the device.

In some examples, device 104 may emit light. In some examples, device 104 may comprise light emitters disposed within. Device 104 may comprise light emitters disposed on a substrate such as a circuit board. In some examples, device 104 may comprise circuit boards populated with LEDs. In some examples, device 104 may comprise lenses or diffusers for emitted light to pass through. In some examples, device 104 may comprise baffles to direct the emitted light.

In some examples, device 104 may be powered from the object, product, or assembly it is installed into. Device 104 may connect to an internal power supply or control module of the product and/or assembly it is integrated into. In some examples, device 104 may be powered with AC or DC voltage. In some examples, device 104 may comprise disposable or rechargeable batteries or single battery. In some examples, device 104 may comprise wires 110 exiting the device to be connected to power. In some examples, device 104 may comprise of multiple other components. In some examples, device 104 may comprise a power supply or LED Driver configured to convert input electrical power to the power required by device 104. In some examples, device 104 may comprise a controller that is configured to control the output of disinfecting light from device 104. The controller may be on the same substrate or circuit board as the light emitters, or it may be remote from the substrate or circuit board with the light emitters. In some examples, device 104 may communicate with sensors. Sensors may be comprised within device 104 or may be remote from device 104 and integrated into a larger assembly such as an appliance. In some examples, device 104 may comprise of user manual controls such as buttons or switches for controlling the disinfecting light output.

In some examples, device 104 may be able to mount onto component 102. In some examples, device 104 may be smaller than component 102. In some examples, device 104 may have an outer diameter that is smaller than the outer diameter of component 102. Device 104 of FIG. 2 may be fixed to component 102. In some examples, device 104 may be removable fixed to component 102. Device 104 may be permanently fixed to component 102. In some examples, device 104 may comprise magnets, suction cups, vacuum sealing device, mounting brackets, etc. Device 104 may be configured to attach to or otherwise be integrated into another product and/or assembly such as an appliance.

In some examples, device 104 may vary in length depending on the application. Device 104 may have a length within the range of 2 in to 24 in including any length in between such as 4 in, 6 in, 8 in, 10 in, 12 in, 14 in, 16 in, 18 in, 20 in, etc. Device 104 may have varying geometry.

In some examples, device 104 may be configured to disinfect the door gasket of a front load washing machine. In some examples, device 104 may be configured to disinfect the drum of a washing machine. Device 104 may be configured to disinfect both the gasket and drum of a washing machine. Device 104 may be configured to be placed inside an appliance. Device 104 may be configured to be placed inside the drum of a washing machine.

In some examples, device 104 may be water sealed. Device 104 may have an Ingress Protection (IP) rating of IP64, IP65, IP66, IP67, IP68, IP69, IP69K, etc.

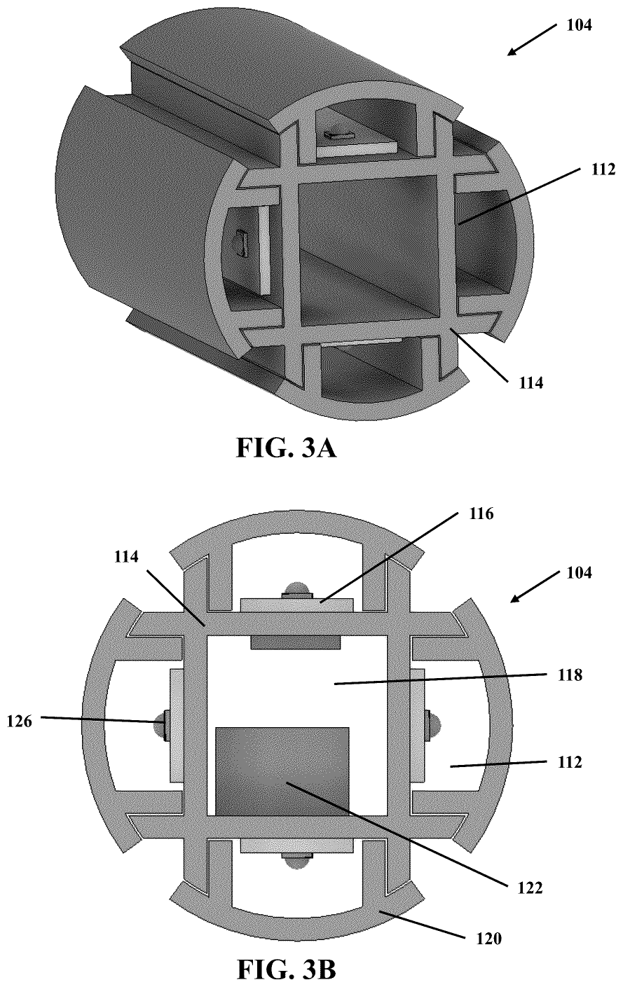

FIG. 3A and FIG. 3B show example details of device 104. Device 104 may be configured to hold light emitters and/or circuit boards. In some examples, device 104 as shown in FIG. 3A may comprise of an internal structure 114. Internal structure 114 may have channels 112 built into it for holding light emitters, substrates and/or circuit boards/substrates disposed with light emitters 116. In some examples, device 104 may comprise an inner channel 118 for mounting electrical devices such as sensor(s), controller(s), and/or power supply or power supplies 122, etc. In some examples, device 104 may comprise a lens or lenses 120 over the light emitters. Lens 120 may be rounded, flat, comprise baffles to direct light, and/or comprise optical features to direct the light emitted from the light emitters. Lens 120 may comprise of design features that allow it to slide or snap into the channels 112 built into internal structure 114.

In some examples, internal structure 114 may have 1, 2, 3, 4, 5, 6, 7, 8, 9, 10, 11, 12 or channels and/or light emitting sides or edges 112. Device 104 may have a minimum of 3 channels and/or light emitting sides or edges 112. Device 104 may have a minimum of 4 channels and/or light emitting sides or edges 112. Device 104 may have the same amount of channels and/or light emitting sides or edges 112 as lenses 120. In some examples, the outer edge of device 104 may be one continuously illuminated surface. In some examples, internal structure 114 may be an extruded profile. Internal structure 114 may have different profile geometry than shown in FIG. 3B. Internal structure 114 may have a length within the range of 2 in to 24 in including any length in between such as 4 in, 6 in, 8 in, 10 in, 12 in, 14 in, 16 in, 18 in, 20 in, etc. In some examples, device 104 may comprise a heat sink. In some examples, internal structure 114 is also a heat sink. Internal structure 114 may comprise features for mounting other components, light emitters, and/or light emitters disposed on substrates such as circuit boards.

In some examples, internal channel 118 may not exist and the center of internal structure 114 may be solid. Internal structure 114 may comprise of heat sinking elements, such as fins, in the internal channel. Heat sinking elements may be located on the other side of the surface of the internal channel 112 that the light emitter 116 is disposed upon. In some examples, internal structure 114 is aluminum. In some examples, internal structure 114 is stainless steel. In some examples, internal structure 114 is made of metal. In some examples, internal structure 114 is made of plastic.

In some examples, power supply 122 may convert AC power to DC power. Power supply 122 may be located between input AC power and the light emitters 116. In some examples, the light emitters 116 receive DC power. Power supply 122 may supply the required power to light emitters 116. In some examples, the power supply is not located within internal channel 118 and is remote from device 104. The power supply may be part of the larger product, assembly, or appliance that device 104 is located within.

In some examples, a controller in communication with device 104 may control a characteristic of the emitted light. In some examples, a characteristic of the emitted light many include one or more of the following: radiometric energy, color, color coordinates, color temperature, brightness, lumen output, mode of operation, wavelength range emitted, powered on or powered off, etc.

In some examples, a sensor in communication with a controller may comprise of one of the following: motion sensor, occupancy sensor, vibration sensor, light sensor, temperature sensor, humidity sensor, etc.

Light emitter may refer to an individual light emitting component, such as an LED, and/or a substrate, such as a circuit board, with light emitters disposed upon it.

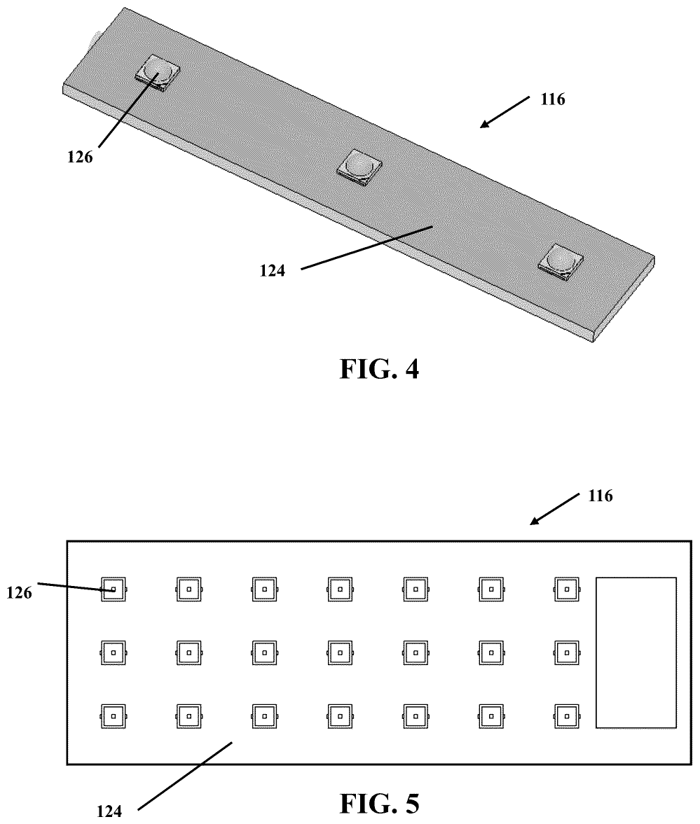

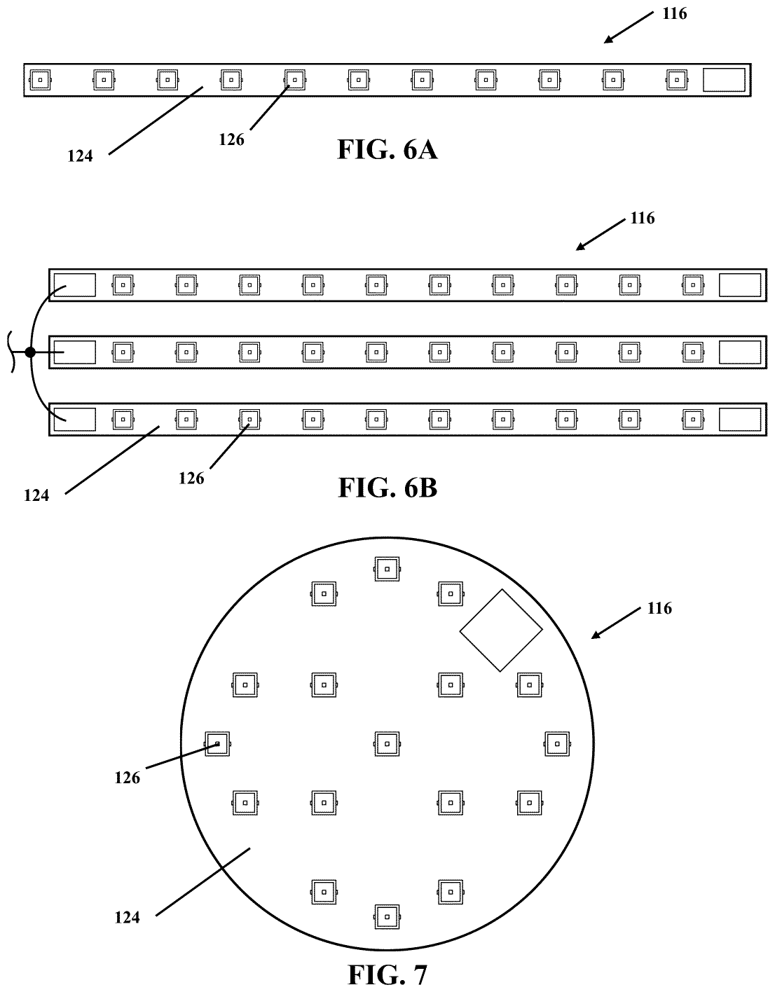

FIGS. 4, 5, 6A and 6B, and 7 show example light emitters 116. In some examples where light emitter 116 comprises light emitters disposed on a substrate 124, such as a circuit board, the substrate 124 may be linear in shape as shown in FIG. 6A. The substrate 124 may be rectangular in shape as shown in FIGS. 4 and 5. The substrate 124 may be circular in shape as shown in FIG. 7. The substrate 124 may have an irregular shape not shown in the FIGS.

In some examples, the substrate 124 may be made of metal. Substrate 124 may be made of aluminum. Substrate 124 may be made of plastic. Substrate 124 may be made of FR4. The substrate may have an associated thickness.

In some examples, the substrate 124 may be designed in a shape that allows it to be used within device 104. Substrate 124 may be installed against the surface of device 104 or within channels 112 of device 104.

In some examples, light emitter 116 may emit light within the range of 380 to 420 nano meters (nm). Light emitter 116 may emit light within the range of 380 to 750 nanometers. Light emitter 116 may emit light within the range of 200 to 400 nanometers. Light emitter 116 may emit light with a peak wavelength within the range of 380 to 420 nm. Light emitter 116 may emit light with a peak wavelength of 405 nm. Light emitter 116 may emit light with a peak wavelength of any value between 375 and 425 nm in increments of 1.

In some examples, light emitters emit light within the ultraviolet range. In some examples, light emitters emit light with a peak wavelength in the ultraviolet range. In some examples, light emitters emit light with a peak wavelength that is not in the ultraviolet range.

In some example light emitter 116 may comprise of a circuit board with one or more light emitters disposed on it. In some examples, light emitter 116 may have LEDs 126 disposed on it. Light emitter 116 may have 1 LED disposed on it. Light emitter 116 may have 2 LEDs disposed on it. Light emitter 116 may have 3, 4, 5, 6, 7, 8, 9 10, 11, 12, 13, 14, 15, 16, 17, 18, 19, 20, 21, 22, 23, or 24 LEDs disposed on it. Light emitter 116 maybe have 12 or more LEDs disposed on it. In some examples, light emitter 116 may comprise a laser.

Light emitter 116 may emit light that is configured to inactivate microorganisms. Light emitter 116 may emit light that is configured to inactivate viruses. Light emitter 116 may emit light that is configured to inactivate microorganisms and viruses on surfaces, in air, and/or within a liquid, such as water. Light emitter 116 may cause microbial inactivation over time. In some examples, higher irradiances are used over shorter periods of time. In some examples, lower irradiances are used over longer periods of time.

In some examples, light emitter 116 may emit light that creates an irradiance on a surface a distance away from the light emitter. In some examples, light emitter 116 may cause an irradiance on a surface of at least 0.01 mW/cm². Light emitter 116 may cause an irradiance on a surface of at least 0.05 mW/cm². Light emitter 116 may cause an irradiance on a surface of at least 0.1 mW/cm². Light emitter 116 may cause an irradiance on a surface of at least 1 mW/cm². Light emitter 116 may cause an average irradiance on a surface of 1 mW/cm². Light emitter 116 may cause an average irradiance on a surface of 0.1 mW/cm². Light emitter 116 may cause an average irradiance on a surface of 0.05 mW/cm². Light emitter 116 may cause an average irradiance on a surface of 0.01 mW/cm². A target irradiance may be at least, greater than, less than, equal to, or any number in between about 0.01 mW/cm² and 10 mW/cm². In some examples an average irradiance is targeted across at least a portion of a surface and not the entire surface. The average irradiance may comprise an average of multiple measurement points taken across at least a portion of the surface. In some examples, irradiance measurements may range from 0 mW/cm² to 100 mW/cm². In some examples, the target average irradiance may be any value within the range of 0.02 mW/cm² to 2 mW/cm². In some examples the average irradiance may be any value within the range of 0.02 mW/cm² to 5 mW/cm².

In some examples, light emitter 116 may emit a minimum amount of radiometric energy measured from at least, greater than, less than, equal to, or any number in between about 10 mW and 2000 mW. Light emitter 116 may emit a radiometric energy sufficient to cause an irradiance on a surface a distance away. The radiometric energy may be sufficient enough to inactivate at least one microorganism's population and/or multiple microorganism populations. In some examples, substrate 124 may comprise multiple light emitters that emit a combined radiometric energy. Substrate 124 may comprise multiple LEDs 126 that emit a combined radiometric energy. In some examples, light emitters 116 may emit a radiometric energy of at least 20 mW measured within the range of 380 to 420 nm. In some examples, light emitters 116 may emit a minimum amount of radiometric energy measured from, at least, greater than, less than, equal to, or any number in between about 375 nm and 425 nm.

In some examples, a dosage measured in J/cm\hat{ }2 is targeted. A dosage may be used as a metric for determining an appropriate irradiance for microbial inactivation over a period of time. A target reduction in microorganism population(s) may be used (e.g., 1- Log 10 reduction, 2- Log 10 reduction, 99% reduction, or the like). In some examples a target dosage may be at least 20 J/cm\hat{ }2. A target dosage may be between 10 and 100 J/cm\hat{ }2. A target dosage may be more than 100 J/cm\hat{ }2.

A dosage may be comprised of an irradiance and exposure time. In some examples, a target exposure time may be at least 1 hour. A target exposure time may be at least 4 hours, at least 6 hours, at least 8 hours, at least 10 hours, at least 12 hours, at least 24 hours, or at least 48 hours. In some examples, exposure may be continuous and not limited to a time period. In some examples, a target exposure time may be calculated continuously from measured usage.

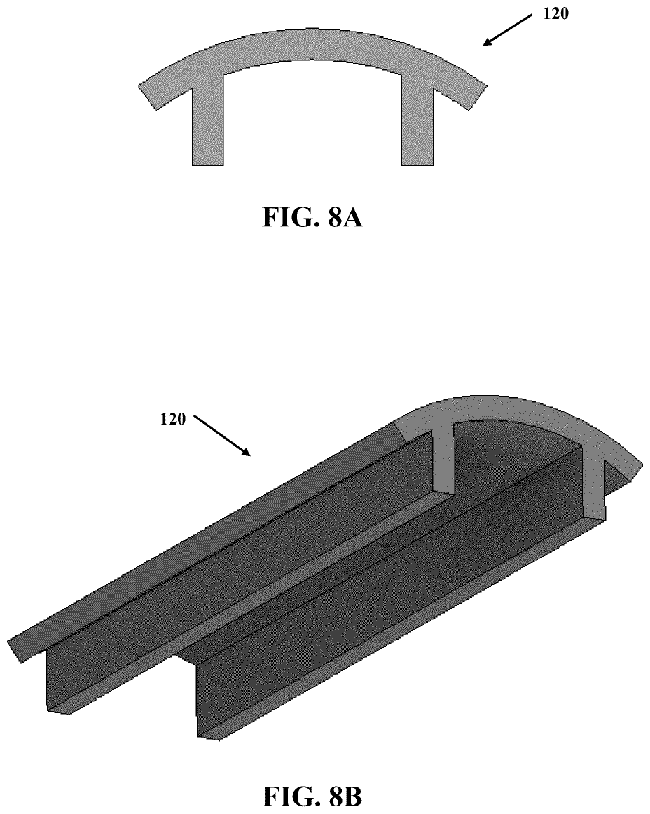

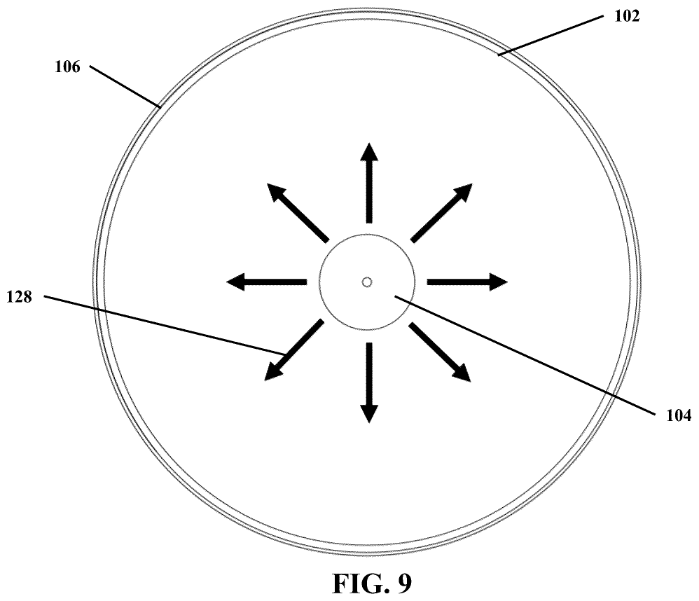

FIGS. 8A and 8B show examples of lens 120. In some examples, lens 120 may be a lens or diffuser for light to pass through. In some examples, lens 120 may be transparent or translucent. In some examples, lens 120 may protect light emitters 116 from water and/or debris. In some examples, lens 120 may transmit light within the range of 380 to 420 nm. Lens 120 may transmit at least 70% of light within the range of 380 to 420 nm. Lens 120 may transmit at least 80% of light within the range of 380 to 420 nm. Lens 120 may transmit at least 90% of light within the range of 380 to 420 nm. Lens 120 may diffuse transmitted light to prevent hot spotting due to point source light emitters such as LEDs 126.

FIG. 9 displays a front facing schematic drawing of device 104 placed in the center of a circular target surface to be disinfected. In some examples, component 106 may be the target surface to be disinfected. In some examples, light is emitted radially from device 104 towards the target surface(s) as shown in FIG. 9.

In some examples, device 104 may emit disinfecting light 360 degrees around the central axis out of the device towards another object, component, or surface. Device 104 may emit light less than 360 degrees around the central axis out of the device. Device 104 may have multiple instances of light each with an associated beam angle. Each associated beam angle may add up to be 360 degrees or less than 360 degrees. An example beam angle may be within a range of 90 to 180 degrees. An example beam angle may be 120 degrees. An example beam angle may be 130 degrees.

In some examples, a photocatalyst may be used to enhance the disinfection. The photocatalyst may be coated on or embedded into the target surface for disinfection. The photocatalyst may be coated on or embedded into a lens. The photocatalyst may be coated on or embedded into a light emitter.

In some examples, the number of light emitters is selected based on the distance between the light emitters and the target surface to be disinfected. In some examples, the distance between the light emitters and the target surface to be disinfected may be between 0.25 in and 24 in. In some examples, the distance between the light emitters and the target surface may be more than 24 in or less than 0.25 in. In some examples, the distance between the target surface and the light emitters is between 6 in and 12 in. In some examples, the distance between the light emitters is between 6 in and 18 in.

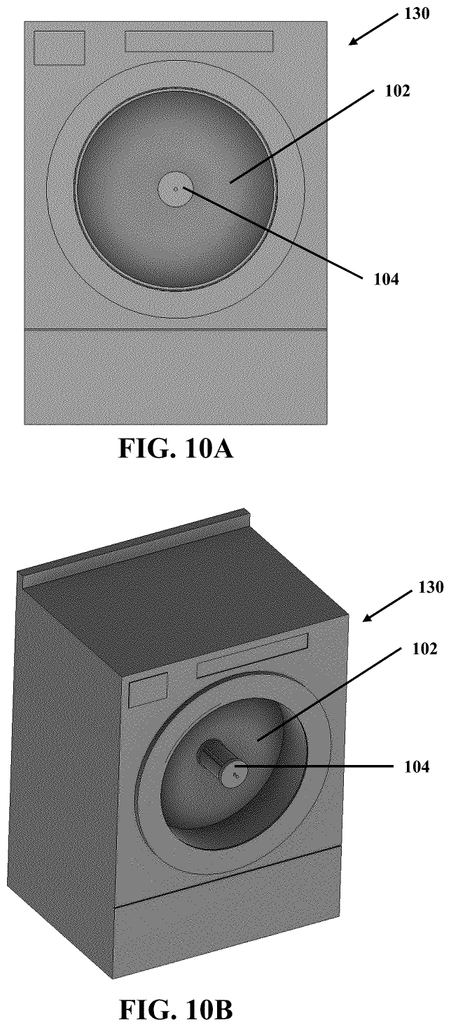

FIG. 10A and FIG. 10B show views of an appliance 130 in which device 104 is installed within. In some examples, appliance 130 may be a front load washing machine. Appliance 130 may be an appliance other than a front load washing machine.

In some examples, device 104 may apply or emit disinfecting light while appliance 130 is running a cleaning cycle. In some examples, device 104 may emit disinfecting light while appliance 130 is not running a cleaning cycle. A cleaning cycle may occur when items are placed inside the appliance for a period of time to be cleaned. In some examples, device 104 may continuously emit disinfecting light. Device 104 may be continuously applying disinfecting light to the gasket of a front load washing machine. Device 104 may be emitting disinfecting light to appliance 130 during a timed cycle of 4, 5, 6, 7, 8, 9, 10, 11 or 12 hours.

Device 104 may stop emitting disinfecting light or power off when motion is detected by a sensor within the system. The sensor may be part of device 104 or may be part of appliance 130.

In some examples, device 104 and/or appliance 130 may comprise a timer that powers off the emitted disinfecting light after a certain period of time. The certain period of time may be directly linked to a dosage requirement. In some examples the period of time may be 4, 5, 6, 7, 8, 9, 10, 11 or 12 hours. The period of time may be linked to a cleaning cycle. In some examples, the period of time may be linked to time between cleaning cycles.

In some examples device 104 may interface with a mobile device or mobile application. In some examples, device 104 may interface with a wireless network. Device 104 may be compatible with controls that allow a user to remotely control the device. In some examples, device 104 may interface with controls directly within an appliance. Device 104 may communicate light output characteristics to a user through a mobile device or application. In some examples, there is an indication that the disinfecting light cycle is over. The indication may include a notification on a mobile application, a message on the appliance, a sound, and/or a change in lighting from the appliance.

In some examples, device 104 may be applied to or installed on or within household or commercial appliances. In some examples, device 104 may be applied to a front load washing machine to disinfect the gasket.

Light emitters may be powered on, emit, output, apply, direct, illuminate, or shine light and/or disinfecting light.

In some examples, device 104 may match the geometry of the object it is installed within. For example, a door on a front load washing machine may be circular in shape, therefore device 104 may have a circular profile.

In some examples, device 104 may comprise reflectors for controlling the path of light or disinfecting light. Device 104 may comprise optics for controlling the path of light or disinfecting light.

In some examples, disinfecting light may need to transmit through component 102 to reach component 106.

In some examples, the disinfecting light emitted from device 104 may prevent odors, may reduce odors, may inactivate odor causing bacteria, and/or may inactivate odor causing microorganisms.

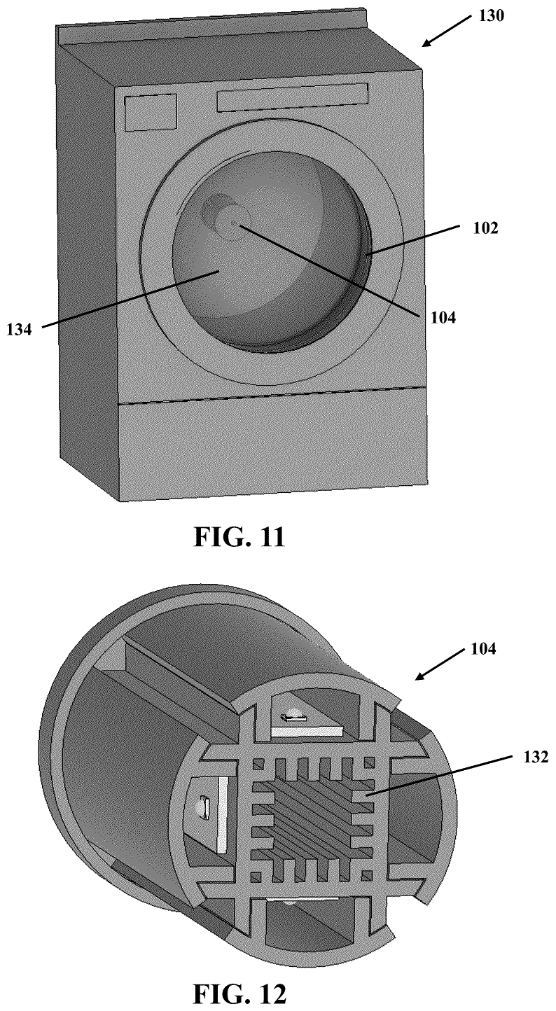

FIG. 11 displays device 104 installed inside appliance 130. Appliance 130 may be a washing machine with an internal drum 134 for holding items placed inside for cleaning, such as clothing. Device 104 may be installed within the drum 134 of a washing machine to disinfect the drum 134 of the washing machine. Device 104 may be configured to disinfect the inner volume of a cylindrical hollow object. In some examples, device 104 remains inside drum 134 continuously. Device 104 may only be placed inside drum 134 in between cleaning cycles of appliance 130 and may be removed during cleaning cycles of appliance 130.

FIG. 12 shows internal structure 112 with heat sinking fins.

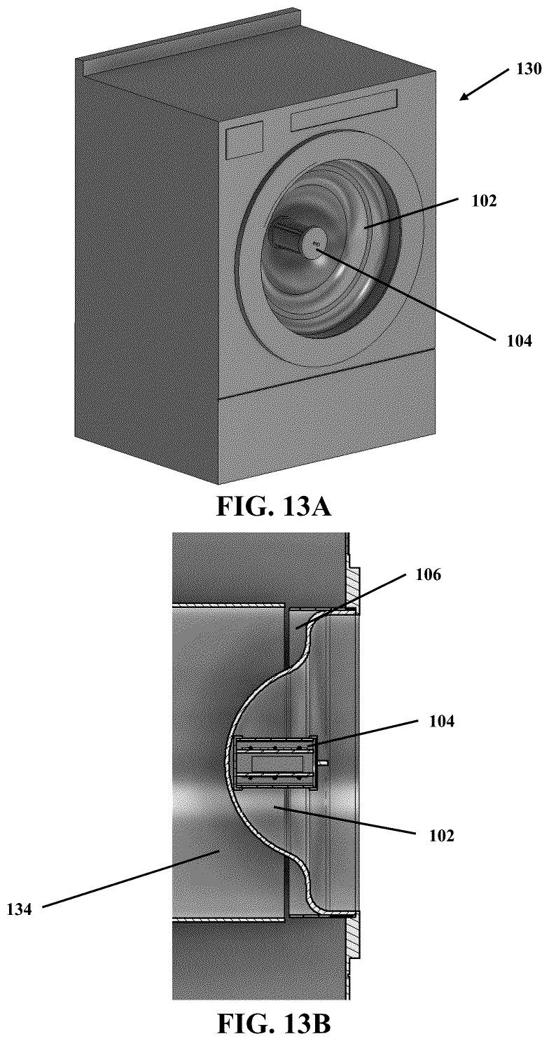

FIG. 13A and FIG. 13B show a different form factor for component 102. In some examples, component 102 may have a volumetric shape that protrudes inward (i.e. domed shape, concave) towards the internal volumetric area of appliance 130. In some examples where appliance 130 is a front load washing machine, this may keep the contents of the drum (i.e. clothes) out of the door and gasket area. This shape may still be acceptable for use with device 104.

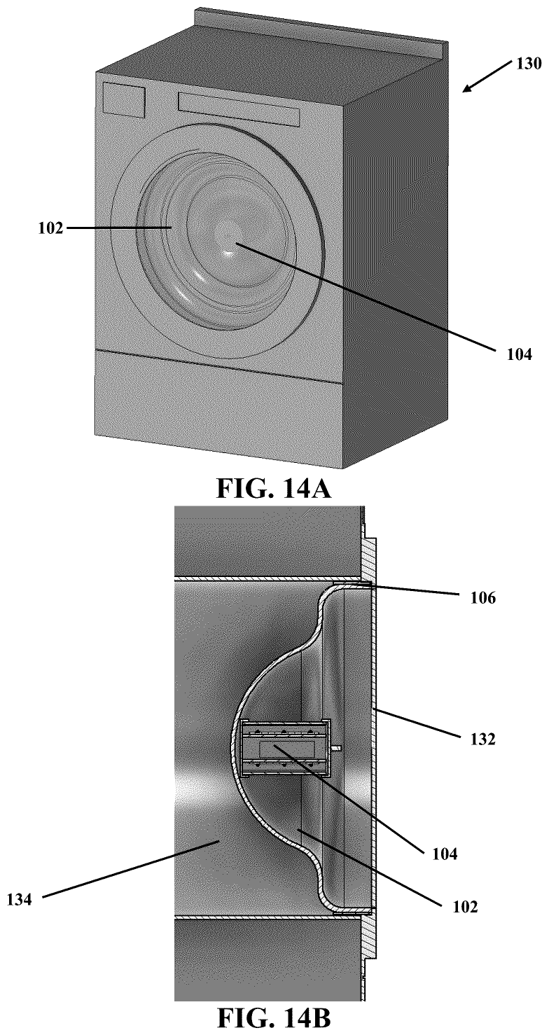

FIG. 14B shows a cross section of FIG. 14A which includes an added layer creating a flat front surface of component 102. In this embodiment, device 104 is installed within an internal volumetric space of component 102. In some examples, there may be an additional layer as part of component 102 (i.e. door) to create a flat surface at the exterior of component 102 and also allow for the shape of component 102 to protrude into the internal area of appliance 130. This creates an inner volumetric space within the component 102 that device 104 may be placed into or within. In some examples, these two layers of component 102 are made of a transparent or translucent material such as glass.

In some examples, there are no opaque components between device 104 and component 102 and/or component 106. This may allow for the disinfecting light emitted from device 104 to contact the target surfaces of component 106 without being blocked. In some examples, there are no opaque components in the direct path of the light emitted from device 104. In some examples, there are no opaque components in the direct path of the light emitted from device 104 to the target surface on appliance 130 and/or on object 106. In some examples, there are transparent or translucent components in the direct path of the light emitted from device 104. In some examples, there are one or more transparent or translucent layers between device 104 and component 106 or a target surface on component 106 or appliance 130. In some examples, component 102 has a length, width, or depth that approximately matches and aligns with the length of device 104.

In some examples, the device disclosed herein may use continuous disinfection. For example, an object or a surface intended to be disinfected may be continuously irradiated by one or more of the light emitter(s) disclosed herein. In some examples, an object or surface may be illuminated for a first percentage of time (e.g., 80% of the time) and not illuminated for a second percentage of time (e.g., 20% of the time), such as when the object or surface is being interacted with by a human, e.g., when running a cleaning cycle, etc. In some examples, an integrated control system may determine that a minimum dosage over a certain period of time has been met for disinfecting purposes and disinfecting light may be turned off to save energy until the period of time expires and resets. In some examples, disinfecting light may be turned off 30% of the time over a specific time period, such as 24 hours, and may still be considered continuous (e.g., 16.8 hours out of 24). Other similar ratios may be possible.

In some examples, the light emitter(s) disclosed herein may use intermittent disinfection. Some examples use intermittent disinfecting techniques where the disinfecting light may be only irradiating an object or surface intended to be disinfected, e.g., a washing machine gasket, for certain period of time. In some examples, disinfecting light may shine on the object or surface intended to be disinfected for 8 hours overnight. In some examples, disinfecting light may shine on the object or surface intended to be disinfection for a period of time between 30 seconds and 8 hours. In some examples, the period of time the object or surface is exposed to the disinfecting light may match up with a specific time required to meet a certain dosage target for the inactivation of a specific microorganism.

As described herein, light used for disinfection may be continuous or intermittent. An object or a surface may be illuminated by disinfecting light, for example, if the object or the surface is not being interacted with (e.g., not being used) by a user. An object or a surface may not be illuminated by disinfecting light, for example, if the object or the surface is being interacted with (e.g., being used) by a user. For example, disinfecting light may be deactivated if a user opens an appliance door (e.g., a washing machine door), etc.

In some examples, one or more of the light sources disclosed herein may pulse disinfecting light. By pulsing the disinfecting light emitter(s) or otherwise reducing its duty cycle below 100%, the dose and exposure may be decreased, and the lifetime of the light emitter(s) may be increased. Pulsed light at high irradiances may be more effective than continuous light at lower irradiances. In some examples, pulsed light may have higher exposure limits compared to a continuous light source. In some examples, pulsed light may be considered to be intermittent because the light will be on and off periodically. In some examples, however, pulsed light may be used continuously and thus may also be considered continuous disinfection due to the length of time that light is pulsed (e.g., light may be pulsed for 24 hours straight).

In some examples, the light emitter(s) may emit light according to a proportion of spectral energy. The proportion of spectral energy may be an amount of spectral energy within a specified wavelength range, i.e., 380-420 nm, divided by a total amount of spectral energy of the light. In some examples, the proportion of spectral energy may be a percentage.

The light emitted from the light emitter(s) may comprise a proportion of a spectral energy of the light, measured in a 380 nanometers (nm) to 420 nm wavelength range, greater than 50%. In some examples, the proportion of spectral energy within the range of 380 to 420 nm may be greater than 70%, greater than 80%, or greater than 90%. In some examples, the proportion of spectral energy within the range of 380 to 420 nm may be approximately 100%. The light may comprise a full width half max (FWHM) emission spectrum of less than 20 nm and centered at a wavelength of approximately 405 nm to concentrate the spectral energy of the light and minimize energy associated with wavelengths that bleed into an ultraviolet wavelength range. The light may provide an irradiance at the surface sufficient to initiate inactivation of microorganisms on the surface.

Different colors of light may be emitted with a percentage (e.g., 20%) of their spectral energy within the wavelength range of 380-420 nm or within a UV wavelength range. In some examples, various colors of light may be emitted with a percentage of 30% to 100% spectral power within the wavelength range of 380-420 nm. For example, a white light containing light across the visible light spectrum from 380-750 nm, may be used for disinfection purposes, with at least 20% of its energy within the wavelength range of 380-420 nm. In some examples the percentage of spectral energy within the wavelength range of 380-420 nm is at least 50%. In some examples the percentage of spectral energy within the wavelength range of 380-420 nm is at least 60%. In some examples the percentage of spectral energy within the wavelength range of 380-420 nm is at least 70%. In some examples the percentage of spectral energy within the wavelength range of 380-420 nm is at least 80%. In some examples the percentage of spectral energy within the wavelength range of 380-420 nm is at least 90%. In some examples the percentage of spectral energy within the wavelength range of 380-420 nm is at least 95%. In some examples the percentage of spectral energy within the wavelength range of 380-420 nm is at least 99%. In some examples the percentage of spectral energy within the wavelength range of 380-420 nm is at least 100%.

In some examples, light emitted from light emitter(s) may be white, may have a color rendering index (CRI) value of at least 70, may have a correlated color temperature (CCT) between approximately 2,500K and 5,000K and/or may have a proportion of spectral energy measured in the 380 nm to 420 nm wavelength range between 10% and 44%. Other colors (e.g., blue, green, red, etc.) may also be used with a minimum percentage of spectral energy (e.g., 20%) within the range of 380-420 nm, which provides the disinfecting energy. In some examples, the white light may include a proportion of spectral energy measured in the 200 nm to 230 nm wavelength range between 0.01% and 2%.

Light emitter(s) may take any light emitter form capable of emitting light or energy e.g., light emitting diode (LED), LEDs with light-converting layer(s), laser, electroluminescent wires, electroluminescent sheets, flexible LEDs, organic light emitting diode (OLED), or a semiconductor die.

In some examples, the light emitters may be LEDs (light emitting diodes) emitting light with a peak wavelength, for example, at least, greater than, less than, equal to, or any number in between about 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, and 425 nm.

In some examples, the light emitters emit at a beam angle of 130 degrees. In other examples, the light emitters emit at a beam angle of, for example, at least, greater than, less than, equal to, or any number in between about 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees.

In some examples, any lens material in the device may comprise an antistatic element to prevent the buildup of particles on it. In some examples the lens may comprise an antistatic coating to prevent the buildup of particles on it.

In some examples, the light emitter(s) may emit disinfecting light. The intensity of the disinfecting light from light emitter(s) may vary based on the angle the disinfecting light is emitted from the light emitter(s). In some examples, disinfecting lighting element may have a beam angle of up to 180 degrees. In some examples, the beam angle may be 60, 120, and/or 130 degrees. The intensity of the disinfecting light may be highest in the center of a beam of disinfecting light emitted from the light emitter(s). In some examples, the intensity may be lower towards the edge of the beam of disinfecting light than the center of the beam. In some examples, the intensity at the edge of a beam of disinfecting light may be 50% of the maximum intensity which may occur in the center of the beam. In some examples, the intensity of the disinfecting light may decrease further from the light emitter(s). The disinfecting light may, for example, have a maximum intensity close to the light emitter(s) and the intensity may decrease as the disinfecting light travels further from the light emitter(s). Due to this, the light emitter(s) may be placed such that there is sufficient light coverage on the target surface, i.e., gasket. The spacing of the light emitter(s) is based upon the distance between the light emitter(s) and the target surface, the radiometric power output of the light emitter(s), and the beam angle of the light emitter. In some examples, the light emitter(s) create a circular light coverage area on the target surface. In some examples, the light emitter(s) will be positioned such that the contour line receiving 50% of the maximum intensity which may occur in the center of the beam on the target surface provided from one light emitter, overlaps with the contour line receiving 50% of the maximum intensity which may occur in the center of the beam on the target surface provided from a separate light emitter such that the areas on the target surface receiving less than 50% of the maximum intensity which may occur in the center of the beam is minimized. In some examples, the overlap between the emitters may be, for example, at least, greater than, less than, equal to, or any number in between about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% and 80% of the maximum intensity.

In some examples, a surface to be disinfected may be in close proximity to a light emitter. In such examples, a device may require more light emitters than would otherwise be necessary for disinfection. The area illuminated by a single light emitter may be limited by a beam angle of the light emitter. The same light emitter may illuminate a larger surface area of the surface to be disinfected if the light emitter is moved further away. Therefore, the device disclosed may need an increased number of light emitters to cover the entire surface area of the surface to be disinfected with disinfecting light, as compared to a further distance. Light emitters may be spaced a distance from the surface to be disinfected. The light emitters may emit a light that spreads outwardly toward the surface at a beam angle. The beam angle may comprise half of an angle of light emitted from the light emitter, in degrees, where the intensity of light is at least 50% of light emitter's maximum emission intensity. In some examples, the light emitter may comprise LEDs and the beam angle may be 130 degrees, e.g., the angle of light emitted from the light emitter where the intensity of light is at least 50% of the maximum emission intensity is 130 degrees. In some examples where light from the light emitter does not possess rational symmetry, the beam angle may be given for two planes at 90 degrees to each other.

A total surface area illuminated by one light emitter may be determined by the beam angle and the distance from the light emitter to the surface intended to be disinfected. A light emitter with a larger beam angle may provide a larger total surface area illuminated by one light emitter. An increased distance between the light emitter and the surface may also increase the total surface area illuminated by one light emitter. The total number of light emitters that may be needed to disinfect the entire surface to be disinfected may be based on the total surface area illuminated by one light emitter. As the distance from the surface intended to be disinfected to the light emitter decreases, the number of light emitters that may be needed to disinfect the surface may increase.

In some examples, a control system may be operatively coupled to the device or system it is utilized in. The example control system may be operative to control operational features of the device such as but not limited to: a duration of illumination, type of light emitter used, exiting light color, light intensity, and/or light irradiance, light powered on/off. The control system may include any now known or later developed processor, microcontroller, system on a chip, computer, server, network device, mesh network device, internet-of-things device, mobile device, etc. The light device may also include at least one sensor coupled to the control system to provide feedback to the control system. In some examples, sensor(s) may sense any parameter of the control environment of the device, motion of a user, motion of structure to which device is coupled, temperature, humidity, light reception, presence and/or level of volatile organic chemicals, air quality and/or air particulates and/or presence of microorganisms on exterior surface, vibration, combinations thereof, etc. Sensor(s) may include any now known or later developed sensing devices for the desired parameter(s). The control system with sensor(s) (and without) can control operation to be continuous or intermittent based on external stimulus, and depending on the application.

In some examples, the control system and/or lights may be wired or wirelessly coupled to the internet (with or without a gateway) and a cloud or on-premises server to control or record data associated with the control system and/or light emitters. In some examples, usage patterns and determinations regarding time-on in different modes, irradiance or dosage thresholds being met may be recorded.

Some microorganisms may respond differently to different wavelengths. In some examples, the control system may adjust the spectrum of the light based on the type of microorganism. For instance, some microorganisms may require high levels of 405 nm light, e.g., >1 mW/cm² for several hours. In some examples, the 405 nm light may be required, for example, at least, greater than, less than, equal to, or any number in between about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 and 72 hours. The same microorganisms may only require 10 uW/cm² at 222 nm, for example, in a smaller time period (minutes) to achieve the same kill. Therefore, it may be beneficial to know the type of microorganism so that the spectrum can be tailored to it. In some examples, the control system may be pre-programed to target specific microorganisms. In some examples, data regarding dosage, irradiance, etc. for a specific microorganism may be input manually. In some examples, the control system or remote server may comprise a database containing optimal spectra for different types of microorganisms.

In some examples, a bioburden sensor may be used to detect the type of microorganism and transmit information to the control system for targeting the microorganism. In some examples, the bioburden sensor may be an autofluorescence sensor, which may comprise a light emitter to cause excitation of the bioburden, and a sensor to measure the resulting emission from the bioburden. This bioburden sensor may interact with the control system or remote database to cause tuning or adjustment of the light's spectrum.

A computing device (e.g., a controller) may be comprised by the device disclosure and may perform the functions of various control systems described herein, and/or any other computer, controller, or processor-based function described herein. The computing device may implement, for example, a control system for control of various lighting parameters, as described herein. In some examples, the computing device, in communication with one or more sensors and one or more lighting devices may implement lighting and/or disinfecting lighting controls based on sensor measurements. In some examples, the computing device may be a microcontroller configured to implement the functions of various control systems described herein.

The computing device may include one or more processors, which may execute instructions of a computer program to perform any of the features described herein. The instructions may be stored in any type of tangible computer-readable medium or memory, to configure. the operation of the processor. As used herein, the term tangible computer-readable storage medium is expressly defined to include storage devices or storage discs and to exclude transmission media and propagating signals. For example, instructions may be stored in a read-only memory (ROM), random access memory (RAM), removable media, such as a Universal Serial Bus (USB) drive, compact disk (CD) or digital versatile disk (DVD), floppy disk drive, or any other desired electronic storage medium. Instructions may also be stored in an attached (or internal) hard drive. The computing device may include one or more input/output devices, such as one or more sensors, lighting devices, display, touch screen, keyboard, mouse, microphone, software user interface, etc. The computing device may include one or more device controllers such as a video processor, keyboard controller, etc. The computing device may also include one or more network interfaces, such as input/output circuits (such as a network card) to communicate with a network such as example network. The network interface may be a wired interface, wireless interface, or a combination thereof. The computing device may comprise one or more timers to measure time. One or more of the elements described above may be removed, rearranged, or supplemented without departing from the scope of the present disclosure.

Various methods, devices, and systems described herein may use a control system to implement various lighting controls in the device disclosed. The control system may be used to control/adjust various aspects of disinfecting light (e.g., dosage, radiant flux, color, time, wavelength, intensity, and/or irradiance). In various examples, the control system may be used to control similar parameters corresponding to other wavelengths of light as well. The other wavelengths of light may correspond to white light, ultraviolet (UV) light, and/or other wavelengths that are not configured for disinfection. In other examples, controls may be implemented to turn off the disinfecting light when an individual opens the appliance.

The control system may comprise the use of sensors. The sensor(s) may comprise, for example, one or more of irradiance sensors, radiant intensity sensors, motion sensors, voice sensors, odor sensors, capacitive touch sensors, magnetic proximity sensors, light sensors, infrared sensors, cameras, ultrasonic sensors, weight sensors, limit switches, and/or any other sensors.

The control system may comprise a timer. The timer may, for example, measure how long disinfecting light has been emitted towards an object. In some examples, the timer may measure the length of time since an appliance was opened/closed. In some examples, enclosures using a timer to turn off the disinfecting lighting when a dosage has been met may also contain indication lighting to make the user aware that the disinfection cycle is complete. In some examples the indication light may be provided by additional lighting elements emitting colors outside of the disinfecting wavelength range, such as green light within the range of 520 to 560 nanometers.

In some examples, a module capable of emitting ultraviolet light may be used as a subcomponent within a device. The module may comprise of one of more of the following: LED PCBA, emitter, emitter package, driver or ballast, control circuitry, safety sensors, lens, reflector, cover, or enclosure. An LED PCBA may be a printed circuit board with surface mount LEDs. The module may also include driving circuitry, for example, to regulate current and voltage going to the LEDs. An emitter may be a UV emission source that is not an LED. A safety sensor may be used to prevent accidental exposure to the UV light. The safety sensor may comprise of an occupancy sensor, a timer, a button, or a control signal from a remote sensor or control system. The module may be enclosed such that UV light does not leak out and is only emitted through the lens.

Light emitters producing ultraviolet or visible light may comprise, for example, an LED, an array of LEDs, a laser, an array of lasers, a vertical cavity surface emitting laser (VCSEL), or an array of VCSELs. Other light emitters that may be used may include, for example, any emitter capable of emitting ultraviolet light including LEDs, fluorescent lamps without phosphor coatings, xenon arc lamps, mercury vapor, short-wave UV lamps made with fused quartz, black lights (fluorescent lamp coated with UVA emitting phosphor), amalgam lamps, natural or filtered sunlight, incandescent lamps with coatings that absorb visible light, gas-discharge (argon, deuterium, xenon, mercury-xenon, metal-halide, arc lamps, planar microcavity microplasma), halogen lamps with fused quartz, solid-state lamps, excimer lamps (such as Krypton Chlorine), etc. In some examples, an LED emitter may comprise at least one semiconductor die and/or at least one semiconductor die packaged in combination with light converting materials. In some examples, the light emitter may be fitted with optical components that may alter the path of the light. (e.g., focus the light into a beam).

In some examples, the light emitter(s) may be populated onto a light module or substrate, i.e., circuit board module or printed circuit board. The light modules may vary in material, shape, size, thickness, flexibility, and otherwise be conformed to specific applications. Base material of the substrate may comprise a variety of materials such as, for example, aluminum, FR-4 (glass-reinforced epoxy laminate material), Teflon, polyimide, or copper.

In some examples, a light emitter or a light module may comprise a conformal coating. The conformal coating may comprise a polymeric film contoured to the light emitter(s), components on the substrate, and/or substrate. The conformal coatings may provide ingress protection from, for example, condensation or other liquids.

In some examples a transparent or translucent surface, such as a lens, may be required as part of the device as a lens or protective material layer. The transparent or translucent surface may allow for 50%-100% transmission of the disinfecting wavelengths. In some examples the materials incident to the disinfecting wavelength selected for the device may have high reflectance of the disinfecting wavelengths in order to increase the intensity/irradiance. The materials may be, for example, matte or glossy white plastics, or materials with mirror like finishes. In some examples, the transparent or translucent surface may allow for 70%-100% relative transmission of the disinfecting wavelengths to the overall visible spectrum wavelengths. In some examples, the transparent or translucent surface may allow for 50%-100% transmission relative to air of the disinfecting wavelengths. In some examples, materials that exhibit fluorescence under disinfecting light are not used due to the reduction in efficacy from absorption of disinfecting wavelengths and emission of longer wavelengths potentially out of the disinfecting wavelength range. In some examples, additives are added to the material to reduce gradual transmission reduction over time due to exposure to high temperatures.

In some examples, it may be desirable to dissipate heat generated by lighting elements or other components of a light emitter as disclosed herein. A decreased operating temperature may increase reliability and lifetime of a device. Heat may affect the peak wavelength and spectrum emitted by the light emitter(s). For example, as temperatures rise, peak wavelengths may shift to longer wavelengths. Similarly, as temperatures decrease, peak wavelengths may shift to shorter wavelengths. Therefore, it may be desirable to constrain the temperature to a certain range in order to maintain a desired peak wavelength or spectrum within some tolerance. In some examples, the light emitter or light module may be coupled to a heatsink. The heatsink may be made out of plastics, ceramics, or metals including, for example, aluminum, steel, or copper. The heatsink may also be made out of a plastic or ceramic material. In some examples the heatsink may be permanently coupled to a light emitter or light module, or otherwise considered a part of the assembly that makes up the light emitter or light module. In some examples the heat sink may be built into the structure the light module is mounted to, such as the internal structure of device 104.

The device disclosed herein may be powered through power outlets, electrical power supplies, batteries or rechargeable batteries mounted in proximity to the appliance, and/or wireless or inductive charging. Where rechargeable batteries are employed, they may be recharged, for example, using AC power or solar panels (not shown), where sufficient sunlight may be available. In some examples, AC power and an AC to DC converter, i.e. LED driver or power supply, may be utilized. In some examples, direct DC power may be utilized when available. In some examples, the device will take in direct DC power from the device or appliance it is installed into, a washing machine for example.

In some examples a system may comprise an appliance comprising a surface for mounting and a target surface for disinfection. The system may comprise a device configured to disinfect an appliance. The device may comprise an internal structure configured to hold one or more light emitters, wherein the one or more light emitters emit disinfecting light with a lighting characteristic and a peak wavelength in a range of 380 to 420 nm. The device may comprise a mounting mechanism configured to mount the device to the surface for mounting. The device may comprise a controller configured to control the output of disinfecting light. The disinfecting light may emit from the device towards the target surface for disinfection. The target surface for disinfection may not be on the device.

In some examples, the device is removably coupled to the surface for mounting.

In some examples, the appliance is a washing machine configured to run a cleaning cycle. The surface for mounting may be a surface on a door of a washing machine or an internal drum of a washing machine. In some examples, the target surface for disinfecting is a door gasket of the washing machine or the internal drum of the washing machine.

In some examples, the door gasket of the washing machine is transparent or translucent and allows light within a range of 380 to 420 nanometers to transmit through it.

In some examples, the disinfecting light is emitted until a dosage threshold is met, wherein the dosage threshold is at least 20 J/cm².

In some examples, the disinfecting light comprises an irradiance on the target surface for disinfection of at least 0.05 mW/cm² and comprises an exposure time period correlating to the target dosage threshold and the irradiance.

In some examples, there are no opaque components in a direct path of the emitted disinfecting light and at least a portion of the target surface for disinfection.

In some examples, a system may further comprise a sensor in communication with the controller and wherein the sensor is a vibration sensor or motion sensor.

In some examples, a device configured to disinfect an appliance may comprise an internal structure configured to hold one or more light emitters, wherein the one or more light emitters emit disinfecting light with a lighting characteristic and a peak wavelength in a range of 380 to 420 nm. The device may comprise a mounting mechanism configured to mount the device to the surface for mounting. The device may comprise a controller configured to control the output of disinfecting light. The disinfecting light may emit from the device towards the target surface for disinfection. The target surface for disinfection may not be on the device.

In some examples, the device may be cylindrical in shape.

In some examples, the disinfecting light emits from a cylindrical device radially 360 degrees around a circular cross-section of the device.

In some examples, a controller is in communication with a timer and the disinfecting light is emitted while the timer runs for a time period and the disinfecting light is not emitted at the end of the time period.

In some examples, the appliance is a washing machine configured to run a cleaning cycle.

In some examples, a controller is in communication with the appliance and controls the disinfecting light such that the disinfecting light is emitted when the cleaning cycle is not active.

In some examples, the light emitter comprises a circuit board disposed with one or more LEDs.

In some examples, the internal structure comprises the controller or an LED driver configured to provide power to the light emitters.

In some examples, the light emitter comprises a circuit board disposed with one or more LEDs and an on-board controller configured to control the output of the LEDs.

In some examples, the light emitters emit a lighting characteristic. In some examples, the lighting characteristic is a radiometric energy output of at least 200 mW.

In some examples, the internal structure comprises a heat sink.

In some examples, the device comprises lenses disposed over each light emitter and configured to transmit at least 70% of the disinfecting light emitting within the wavelength range of 380 to 420 nanometers.

In various examples described herein, light at a specified wavelength or wavelength range may correspond to light which has a maximum emitted energy/power/energy spectral density/power spectral density approximately at the specified wavelength or within the specified wavelength range, with reasonable variations (e.g., +5 nm, +10 nm, etc.).

The above discussed embodiments are simply examples, and modifications may be made as desired for different implementations. For example, steps and/or components may be subdivided, combined, rearranged, removed, and/or augmented; performed on a single device or a plurality of devices; performed in parallel, in series; or any combination thereof. Additional features may be added.

1. A system comprising:
an appliance comprising a surface for mounting and a target surface for disinfection; and
a device configured to disinfect an appliance comprising:
an internal structure configured to hold one or more light emitters, wherein the one or more light emitters emit disinfecting light with a lighting characteristic and a peak wavelength in a range of 380 to 420 nm;
a mounting mechanism configured to mount the device to the surface for mounting; and
a controller configured to control the output of the disinfecting light.
wherein the disinfecting light emits from the device towards the target surface for disinfection, and wherein the target surface is not on the device.

2. The system of claim 1, wherein the device is removably coupled to the surface for mounting.

3. The system of claim 1, wherein:
the appliance is a washing machine configured to run a cleaning cycle;
the surface for mounting is a surface on a door of the washing machine or an internal drum of the washing machine; and
the target surface for disinfecting is a door gasket of the washing machine or the internal drum of the washing machine.

4. The system of claim 3, wherein the door gasket of the washing machine is transparent or translucent and allows light within a range of 380 to 420 nanometers to transmit through it.

5. The system of claim 1, wherein the disinfecting light is emitted until a dosage threshold is met, wherein the dosage threshold is at least 20 J/cm².

6. The system of claim 5, wherein the disinfecting light:
comprises an irradiance on the target surface for disinfection of at least 0.05 mW/cm²; and
comprises an exposure time period correlating to the target dosage threshold and the irradiance.

7. The system of claim 1, wherein there are no opaque components in a direct path of the emitted disinfecting light and at least a portion of the target surface for disinfection.

8. The system of claim 1, further comprising a sensor in communication with the controller, wherein the sensor is a vibration sensor or a motion sensor.

9. A device configured to disinfect an appliance comprising:
an internal structure configured to hold one or more light emitters, wherein the one or more light emitters emit disinfecting light with a lighting characteristic and a peak wavelength in a range of 380 to 420 nm;
a mounting mechanism configured to mount the device to a surface for mounting; and
a controller configured to control the output of the disinfecting light,
wherein the disinfecting light emits from the device towards a target surface, and wherein the target surface is not on the device.

10. The device of claim 9, wherein the device is cylindrical in shape.

11. The device of claim 10, wherein the disinfecting light emits from the device radially 360 degrees around a circular cross-section of the device.

12. The device of claim 9, wherein the controller is in communication with a timer, and wherein the disinfecting light is emitted while the timer runs for a time period and the disinfecting light is not emitted at the end of the time period.

13. The device of claim 9, wherein the appliance is a washing machine configured to run a cleaning cycle.

14. The device of claim 13, wherein the controller is in communication with the appliance and controls the disinfecting light such that the disinfecting light is emitted when the cleaning cycle is not active.

15. The device of claim 9, wherein the light emitter comprises a circuit board disposed with one or more LEDs.

16. The device of claim 9, wherein the internal structure comprises:
the controller; or
an LED driver configured to provide power to the light emitters.

17. The device of claim 15, wherein the circuit board comprises an on-board controller configured to control the output of the LEDs.

18. The device of claim 9, wherein the lighting characteristic is a radiometric energy output of at least 200 mW.

19. The device of claim 9, wherein the internal structure comprises a heat sink.

20. The device of claim 9, further comprising lenses disposed over each light emitter and configured to transmit at least 70% of the disinfecting light emitting within the wavelength range of 380 to 420 nanometers.
